(19) United States
(12) Patent Application Publication
(10) Pub. No.: US 2015/0368429 A1
(43) Pub. Date: Dec. 24, 2015

(54) POLYESTER MODIFIER COMPOSITION FOR CELLULOSE ESTER RESIN, CELLULOSE ESTER OPTICAL FILM, AND POLARIZING PLATE PROTECTIVE FILM

(71) Applicant: DIC CORPORATION, Tokyo (JP)

(72) Inventors: Takanori Ohtsubo, Ichihara-shi (JP); Yusuke Tajiri, Ichihara-shi (JP); Hiroshi Yoshimura, Ichihara-shi (JP)

(21) Appl. No.: 14/762,643

(22) PCT Filed: Jan. 21, 2014
(86) PCT No.: PCT/JP2014/051075

§ 371 Date: Jul. 22, 2015

(30) Foreign Application Priority Data
Jan. 25, 2013 (JP) 2013-012083

(51) Int. Cl. C08K 5/11; C09D 101/12; C08L 1/12; C07C 69/604

(52) U.S. Cl. CPC C08K 5/11; C07C 69/604; C09D 101/12; C08L 1/12

(57) ABSTRACT

There is provided a polyester modifier composition for a cellulose ester resin, including: a polyester resin produced by a reaction between a diol and 1,2-dicarboxycyclohexane. The modifier composition has a number-average molecular weight (Mn) in the range of 350 to 2,000 as measured by gel permeation chromatography (GPC). The polyester resin has a molecular weight of less than 350 constitutes 5% or less by mass of the modifier composition.

### TECHNICAL FIELD

The present invention relates to a polyester modifier composition for an optical film having high dimensional stability, a cellulose ester optical film formed from the modifier composition, and a polarizing plate protective film formed from the modifier composition.

### BACKGROUND ART

In recent years, because of their space-saving and energy-saving characteristics, liquid crystal displays (LCDs) have been increasingly used in TV sets, personal computers, and mobile phones. With the increasing demand for LCDs, the supply of LCDs is increasing. This makes it important to improve the quality, such as surface physical properties, of optical films for protecting LCD polarizers (polarizing plate protective films).

One of the characteristics required for LCDs is visibility. Improvements in visibility essentially require the dimensional stability of display screens of LCDs, particularly the dimensional stability of polarizing plate protective films constituting the outermost layers of polarizing plates, more specifically, dimensional resistance to long-term degradation or dimensional resistance to heat. The polarizing plate protective films of LCDs are mainly cellulose ester films. Cellulose ester films have problems of dimensional changes due to heat generated by backlight LEDs.

It is known that triphenyl phosphate (TPP) is contained in some cellulose ester films in order to improve the dimensional stability of polarizing plate protective films. It is also known that a cellulose ester film contains an organic acid ester compound containing a polyhydric alcohol ester composed of an aliphatic polyhydric alcohol and at least one monocarboxylic acid (see, for example, Patent Literature 1). However, optical films are becoming thinner and more susceptible to environmental influences. Thus, it is difficult for the cellulose ester film described in Patent Literature 1 to have sufficient dimensional stability.

A technique for forming a layer for maintaining the dimensions on an optical film by coextrusion in the production of the optical film is also known (see, for example, Patent Literature 2). However, this technique has a problem of complex production lines.

### CITATION LIST

### Patent Literature

PTL 1: Japanese Unexamined Patent Application Publication No. 2004-323749

PTL 2: Japanese Unexamined Patent Application Publication No. 2012-179731

### SUMMARY OF INVENTION

### Technical Problem

It is an object of the present invention to provide a polyester modifier composition for an optical film having high dimensional stability, a cellulose ester optical film formed from the modifier composition, and a polarizing plate protective film formed from the polyester modifier composition, without complex production lines.

### Solution to Problem

As a result of extensive studies, the present inventors completed the present invention by finding that an optical film having high dimensional stability can be produced without complex production lines by using a modifier composition for a cellulose ester resin, the modifier composition containing a polyester resin produced by a reaction between a diol and a dicarboxylic acid, the modifier composition having a molecular weight in a particular range, the modifier composition containing a smaller amount of polyester resin having a low molecular weight.

The present invention provides a polyester modifier composition for a cellulose ester resin, the polyester modifier composition containing a polyester resin produced by a reaction between a diol and a dicarboxylic acid, wherein the modifier composition has a number-average molecular weight (Mn) in the range of 350 to 2,000 as measured by gel permeation chromatography (GPC), and a polyester resin having a molecular weight of less than 350 constitutes 5% or less by mass of the modifier composition.

The present invention also provides a cellulose ester optical film that contains the polyester resin modifier composition for a cellulose ester resin and a cellulose ester resin.

The present invention also provides a polarizing plate protective film, formed by casting a resin solution on a metal support, the resin solution produced by dissolving the polyester resin modifier composition for a cellulose ester resin and a cellulose ester resin in an organic solvent, and then evaporating the organic solvent to dry the resin solution.

### Advantageous Effects of Invention

The present invention can provide a polyester modifier composition for a cellulose ester resin from which an optical film having high dimensional stability can be formed. The modifier can be used to form optical films, such as polarizing plate protective films, optical compensation films, and retardation films.

### DESCRIPTION OF EMBODIMENTS

A polyester modifier composition for a cellulose ester resin according to the present invention is a polyester modifier composition for a cellulose ester resin, comprising: a polyester resin produced by a reaction between a diol and a dicarboxylic acid, wherein the modifier composition has a number-average molecular weight (Mn) in the range of 350 to 2,000 as measured by gel permeation chromatography (GPC), and a polyester resin having a molecular weight of less than 350 constitutes 5% or less by mass of the modifier composition. A (Mn) of less than 350 as measured by GPC unfavorably results in an increased amount of volatile component in the optical film and low dimensional stability and heat resistance of the optical film. A (Mn) of more than 2,000 as measured by (GPC) unfavorably results in low compatibility with an optical film substrate, which causes cloudiness of the film. The (Mn) as measured by (GPC) preferably ranges from 500 to 1,800, more preferably 500 to 1,700.

When a polyester resin having a molecular weight of less than 350 constitutes more than 5% by mass of a modifier composition for a cellulose ester resin according to the present invention, this unfavorably results in an optical film having low dimensional stability. This amount is ideally 0% by mass and is preferably 3% or less by mass from a practical point of view with respect to the production of the modifier.

The amount of polyester resin having a (Mn) of more than 2,000 is preferably 1% or less by mass in order to maintain the transparency of the optical film.

A modifier composition for a cellulose ester resin according to the present invention may have any structure and may be produced by any method, provided that the modifier composition contains a polyester resin produced by a reaction between a diol and a dicarboxylic acid and has a (Mn) in the range of 350 to 2,000, and that a polyester resin having a molecular weight of less than 350 constitutes 5% or less by mass of the modifier composition. For example, 1) a modifier for a cellulose ester resin formed of a composition having a (Mn) in the range of 350 to 2,000 in which a polyester resin having a molecular weight of less than 350 constitutes 5% or less by mass of the composition may be produced by adjusting the reaction conditions for a reaction between a diol and a dicarboxylic acid, or 2) a modifier for a cellulose ester resin formed of a composition finally having a (Mn) in the range of 350 to 2,000 in which a polyester resin having a molecular weight of less than 350 constitutes 5% or less by mass of the composition may be produced by producing a polyester resin composition by a reaction between a diol and a dicarboxylic acid and removing a low-molecular-weight polyester resin, more specifically a polyester resin having a molecular weight of less than 350, from the polyester resin composition by one of various methods. Among these, the method 2) is preferred because of its simplicity.

The method for removing the low-molecular-weight polyester resin is not particularly limited and may be an evaporation method with a thin-film distillation apparatus, a column adsorption method, or a solvent separation and extraction method. Among these, an evaporation method with a thin-film distillation apparatus is preferred because this method has no detrimental effects, such as an increase in molecular weight due to transesterification of a mixture of polyester resins having different molecular weights or a decomposition reaction or coloring due to thermal history, and can be performed in a short time.

The number-average molecular weight (Mn) is a polystyrene equivalent molecular weight based on GPC measurement. The conditions for GPC measurement are as follows:

### [GPC Measurement Conditions]

Measuring apparatus: “HLC-8220 GPC” manufactured by Tosoh Corporation

Column: Guard column “HHR-H” manufactured by Tosoh Corporation (6.0 mm I.D.×4 cm)+“TSK-GEL GMHHR-N” manufactured by Tosoh Corporation (7.8 mm I.D.×30 cm)+“TSK-GEL GMHHR-N” manufactured by Tosoh Corporation (7.8 mm I.D.×30 cm)+“TSK-GEL GMHHR-N” manufactured by Tosoh Corporation (7.8 mm I.D.×30 cm)+“TSK-GEL GMHHR-N” manufactured by Tosoh Corporation (7.8 mm I.D.×30 cm)

Detector: ELSD (“ELSD2000” manufactured by Alltech)

Data processing: “GPC-8020 model II data analysis version 4.30” available from Tosoh Corporation

Measurement Conditions:

| | |
|---|---|
| Column temperature | 40° C. |
| Developing solvent | Tetrahydrofuran (THF) |
| Flow rate | 1.0 ml/min |

Sample: A tetrahydrofuran solution having a resin solid content of 1.0% by mass and subjected to microfilter filtration (5 μl).

Standard samples: The following monodisperse polystyrenes having known molecular weights were used in accordance with a measurement manual of the “GPC-8020 model II data analysis version 4.30”.

### (Monodisperse Polystyrene)

“A-500” manufactured by Tosoh Corporation
“A-1000” manufactured by Tosoh Corporation
“A-2500” manufactured by Tosoh Corporation
“A-5000” manufactured by Tosoh Corporation
“F-1” manufactured by Tosoh Corporation
“F-2” manufactured by Tosoh Corporation
“F-4” manufactured by Tosoh Corporation
“F-10” manufactured by Tosoh Corporation
“F-20” manufactured by Tosoh Corporation
“F-40” manufactured by Tosoh Corporation
“F-80” manufactured by Tosoh Corporation
“F-128” manufactured by Tosoh Corporation
“F-288” manufactured by Tosoh Corporation
“F-550” manufactured by Tosoh Corporation

In the present invention, the amount of polyester resin having a molecular weight of less than 350 in the modifier composition is determined from a chart obtained under the GPC measurement conditions.

Specific examples of a polyester modifier composition for a cellulose ester resin according to the present invention preferably include the following polyester modifier compositions.

1) A polyester modifier composition containing a polyester resin (A1) produced by a reaction between an aliphatic diol (a1) and an aliphatic dicarboxylic acid (a2).

2) A polyester modifier composition containing a polyester resin (A2) produced by a reaction between an aliphatic diol (a1) and an aromatic dicarboxylic acid (a3).

The polyester modifier (A1) and polyester modifier (A2) will be described in detail below.

Preferred examples of the aliphatic diol (a1) for use in the production of the polyester resin (A1) include aliphatic diols having 2 to 4 carbon atoms. Examples of the aliphatic diols having 2 to 4 carbon atoms include ethylene glycol, 1,2-propylene glycol, 1,3-propanediol, 2-methylpropanediol, 1,2-butanediol, 1,3-butanediol, 1,4-butanediol, and 2,3-butanediol. Among these, ethylene glycol is preferred because an optical film having high dimensional stability can be produced, and the resulting polyester modifier has high bleeding resistance at high temperature and humidity and can impart sufficient moisture permeability resistance to optical films. These aliphatic diols (a1) may be used alone or in combination.

Preferred examples of the aliphatic dicarboxylic acid (a2) include aliphatic dicarboxylic acids having 2 to 8 carbon atoms. Examples of the aliphatic dicarboxylic acids having 2 to 6 carbon atoms include oxalic acid (the number of carbon atoms: 2; The number in parentheses refers to the number of carbon atoms of the molecule. The same applies hereinafter.), malonic acid (3), succinic acid (4), glutaric acid (5), adipic acid (6), maleic acid (4), fumaric acid (4), 1,2-dicarboxycyclohexane (8), and 1,2-dicarboxycyclohexene (8). Among these, succinic acid, adipic acid, or 1,2-dicarboxycyclohexane is preferred because an optical film having high dimensional stability can be produced, and the resulting polyester modifier has high bleeding resistance at high temperature and humidity and can impart sufficient moisture permeability resistance to optical films. These aliphatic dicarboxylic acids (a2) may be used alone or in combination.

The aliphatic dicarboxylic acid (a2) having 2 to 8 carbon atoms may be a carboxylic acid derivative, such as an ester, acid chloride, or acid anhydride, of the aliphatic dicarboxylic acid, instead of the aliphatic dicarboxylic acid. The carboxylic acid derivatives may be used alone or in combination.

The polyester resin (A1) may also preferably be a polyester resin having a blocked terminal carboxy group that is produced by producing a polyester resin having a terminal carboxy group from the aliphatic diol (a1) and the aliphatic dicarboxylic acid (a2) and reacting the polyester resin having a carboxy group with a monoalcohol (a4) because the polyester resin having a blocked terminal carboxy group serves as a polyester modifier composition for a cellulose ester resin from which an optical film having high moisture permeability resistance can be produced.

Among modifier compositions for a cellulose ester resin containing the polyester resin (A1), a modifier composition containing a polyester resin having a blocked terminal carboxy group produced by a reaction between the aliphatic diol (a1), the aliphatic dicarboxylic acid (a2), and the monoalcohol (a4) may be preferred because the modifier composition serves as a polyester modifier composition for a cellulose ester resin from which an optical film having high moisture permeability resistance can be produced. The polyester resin produced by a reaction between the aliphatic diol (a1), the aliphatic dicarboxylic acid (a2), and the monoalcohol (a4) may be produced by charging the aliphatic diol (a1), the aliphatic dicarboxylic acid (a2), and the monoalcohol (a4) in a reaction system at the same time and reacting them or by producing a polyester resin having a terminal carboxy group from the aliphatic diol (a1) and the aliphatic dicarboxylic acid (a2) and reacting the polyester resin having a carboxy group with the monoalcohol (a4).

Among modifier compositions for a cellulose ester resin containing the polyester resin (A1), a modifier composition containing a polyester resin having a blocked terminal hydroxy group produced by a reaction between the aliphatic diol (a1), the aliphatic dicarboxylic acid (a2), and a monocarboxylic acid (a5) may be preferred because the modifier composition serves as a polyester modifier composition for a cellulose ester resin from which an optical film having high moisture permeability resistance can be produced. The polyester resin produced by a reaction between the aliphatic diol (a1), the aliphatic dicarboxylic acid (a2), and the monocarboxylic acid (a5) may be produced by charging the aliphatic diol (a1), the aliphatic dicarboxylic acid (a2), and the monocarboxylic acid (a5) in a reaction system at the same time and reacting them or by producing a polyester resin having a terminal hydroxy group from the aliphatic diol (a1) and the aliphatic dicarboxylic acid (a2) and reacting the polyester resin having a hydroxy group with the monocarboxylic acid (a5).

The monoalcohol (a4) preferably has 4 to 9 carbon atoms, for example. Examples of monoalcohols having 4 to 9 carbon atoms include 1-butanol, 2-butanol, isobutanol, t-butanol, 1-pentanol, isopentyl alcohol, tert-pentyl alcohol, cyclopentanol, 1-hexanol, cyclohexanol, 1-heptanol, 1-octanol, 2-ethyl-1-hexanol, isononyl alcohol, and 1-nonyl alcohol. Among these, 1-butanol or 1-hexanol is preferred because an optical film having high dimensional stability can be produced, the resulting polyester modifier composition has high bleeding resistance at high temperature and humidity and can impart sufficient moisture permeability resistance to optical films, and the resulting optical film has a low retardation value (Rth) in the film thickness direction.

The monocarboxylic acid (a5) preferably has 4 to 9 carbon atoms, for example. Examples of monocarboxylic acids having 4 to 9 carbon atoms include butanoic acid, pentanoic acid, hexanoic acid, heptanoic acid, octanoic acid, 2-ethylhexyl acid, and nonanoic acid. Among these, butanoic acid is preferred because an optical film having high dimensional stability can be produced, the resulting polyester modifier composition has high bleeding resistance at high temperature and humidity and can impart sufficient moisture permeability resistance to optical films, and the resulting optical film has a low retardation value (Rth) in the film thickness direction.

Among the polyester resins (A1), the polyester resin produced from an aliphatic diol having 2 to 4 carbon atoms, an aliphatic dicarboxylic acid having 2 to 8 carbon atoms, and a monoalcohol having 4 to 9 carbon atoms and/or a monocarboxylic acid having 4 to 9 carbon atoms may be the following polyester resin.

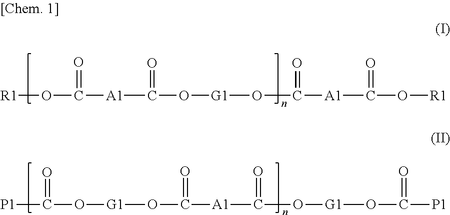

[In the formulae (I) and (II), R1 independently denotes an alkyl group having 4 to 9 carbon atoms, and P1 independently denotes an alkyl group having 3 to 8 carbon atoms. G1 independently denotes an alkylene group having 2 to 4 carbon atoms. A1 independently denotes an alkylene group having 1 to 6 carbon atoms or indicates that two adjacent carbonyl carbons are directly bonded together. n is an integer in the range of 1 to 9.]

In the general formulae (I) and (II), n preferably ranges from 1 to 8.

For example, the polyester resin (A1) can be produced by an esterification reaction between (a1), (a2), and optionally (a4) or (a5) at a temperature in the range of 180° C. to 250° C. for 10 to 25 hours, if necessary, in the presence of an esterification catalyst. The conditions for the esterification reaction, such as temperature and time, are not particularly limited and may be appropriately determined.

Examples of the esterification catalyst include, but are not limited to, titanium catalysts, such as tetraisopropyl titanate and tetrabutyl titanate; tin catalysts, such as dibutyltin oxide; and organic sulfonic acid catalysts, such as p-toluenesulfonic acid.

The amount of the esterification catalyst to be used may be appropriately determined and, in general, preferably ranges from 0.001 to 0.1 parts by mass per 100 parts by mass of (a1), (a2), and (a4) and/or (a4) in total.

The degree of dispersion (Mw/Mn) of the polyester resin (A1) preferably ranges from 1.0 to 3.0, more preferably 1.0 to 1.5. When the polyester resin (A1) has a degree of dispersion in this range, the resulting modifier composition has high compatibility with cellulose ester resins and low volatility.

The degree of dispersion refers to (Mw/Mn) calculated by dividing the weight-average molecular weight (Mw) by the number-average molecular weight (Mn). Mw and Mn are polystyrene equivalents measured by gel permeation chromatography (GPC) using tetrahydrofuran (THF) as an eluent.

The polyester resin (A1) preferably has a hydroxyl value in the range of 0 to 20 mgKOH/g, more preferably 0 to 10. The polyester resin (A1) preferably has an acid value in the range of 0 to 1 mgKOH/g, more preferably 0 to 0.5. Thus, the polyester resin (A1) preferably has a hydroxyl value in the range of 0 to 20 mgKOH/g and an acid value in the range of 0 to 1.0 mgKOH/g and more preferably has a hydroxyl value in the range of 0 to 10 and an acid value in the range of 0 to 0.5.

The acid value results from a polyester resin having a terminal carboxy group that can be produced by a reaction between the aliphatic diol (a1) and the aliphatic dicarboxylic acid (a2). In order to impart good moisture permeability resistance to an optical film and maintain the stability of the polyester resin (A1) itself, the amount of polyester resin having a terminal carboxy group is preferably minimized, and as a criterion the acid value is preferably in the range described above.

The hydroxyl value may result from hydroxy groups not blocked with the monocarboxylic acid (a5) among terminal hydroxy groups of a polyester resin that can be produced by a reaction between the aliphatic diol (a1) and the aliphatic dicarboxylic acid (a2); an aliphatic polyester resin having one terminal hydroxy group that can be produced by a reaction between the aliphatic diol (a1) and the aliphatic dicarboxylic acid (a2); and/or unreacted hydroxy groups of the aliphatic diol (a1). Because the hydroxy group has a high affinity for water, the hydroxyl value is preferably in the range described above also in order to maintain the moisture permeability resistance of the resulting film.

The polyester resin (A2) [a modifier synthesized essentially from the aliphatic diol (a1) and the aromatic dicarboxylic acid (a3)] will be described below.

Among the aliphatic diols (a1), propylene glycol is preferred because an optical film having high dimensional stability can be produced, and propylene glycol has high bleeding resistance at high temperature and humidity and can impart sufficient moisture permeability resistance to optical films.

Preferred examples of the aromatic dicarboxylic acid (a3) for use in the production of the polyester resin (A2) include aromatic (anhydrous) dicarboxylic acids having 8 to 12 carbon atoms and esters thereof. Examples of the aromatic carboxylic acids include (anhydrous) dicarboxylic acids having an aromatic ring structure, such as a benzene ring structure or a naphthalene ring structure, and esters thereof, for example, orthophthalic acid, isophthalic acid, terephthalic acid, phthalic anhydride, 1,4-naphthalenedicarboxylic acid, 2,3-naphthalenedicarboxylic acid, 2,6-naphthalenedicarboxylic acid, 2,7-naphthalenedicarboxylic acid, 1,8-naphthalenedicarboxylic acid, esters and acid chlorides thereof, and an acid anhydride of 1,8-naphthalenedicarboxylic acid. These aromatic carboxylic acids may be used alone or in combination.

Among the aromatic dicarboxylic acids (a3), at least one selected from the group consisting of phthalic anhydride, orthophthalic acid, dimethyl orthophthalate, and dimethyl terephthalate is preferred because the resulting polyester modifier composition has high bleeding resistance at high temperature and humidity and can impart sufficient moisture permeability resistance to optical films.

The polyester resin (A2) can be produced by the same method as the polyester resin (A1).

Among modifier compositions for a cellulose ester resin containing the polyester resin (A2), a modifier composition containing a polyester resin having a blocked terminal carboxy group produced by a reaction between the aliphatic diol (a1), the aromatic dicarboxylic acid (a3), and the monoalcohol (a4) may also be preferred because the modifier composition serves as a polyester modifier composition for a cellulose ester resin from which an optical film having high moisture permeability resistance can be produced. The polyester resin produced by a reaction between the aliphatic diol (a1), the aromatic dicarboxylic acid (a3), and the monoalcohol (a4) may be produced by charging the aliphatic diol (a1), the aromatic dicarboxylic acid (a3), and the monoalcohol (a4) in a reaction system at the same time and reacting them or by producing a polyester resin having a terminal carboxy group from the aliphatic diol (a1) and the aromatic dicarboxylic acid (a3) and reacting the polyester resin having a carboxy group with the monoalcohol (a4).

Among modifier compositions for a cellulose ester resin containing the polyester resin (A2), a modifier composition containing a polyester resin having a blocked terminal hydroxy group produced by a reaction between the aliphatic diol (a1), the aromatic dicarboxylic acid (a3), and a monocarboxylic acid (a5) may be preferred because the modifier composition serves as a polyester modifier composition for a cellulose ester resin from which an optical film having high moisture permeability resistance can be produced. The polyester resin produced by a reaction between the aliphatic diol (a1), the aromatic dicarboxylic acid (a3), and the monocarboxylic acid (a5) may be produced by charging the aliphatic diol (a1), the aromatic dicarboxylic acid (a3), and the monocarboxylic acid (a5) in a reaction system at the same time and reacting them or by producing a polyester resin having a terminal hydroxy group from the aliphatic diol (a1) and the aromatic dicarboxylic acid (a3) and reacting the polyester resin having a hydroxy group with the monocarboxylic acid (a5).

Although the monocarboxylic acid (a5) may be the monocarboxylic acid having an aliphatic structure described above, the monocarboxylic acid (a5) is preferably a monocarboxylic acid having an aromatic skeleton, more preferably a monocarboxylic acid having 7 to 11 carbon atoms and an aromatic skeleton, because the modifier can serve as an additive agent for an optical film that exhibits satisfactory retardation. Examples of the monocarboxylic acid having 7 to 11 carbon atoms and an aromatic skeleton include benzoic acid, dimethylbenzoic acid, trimethylbenzoic acid, tetramethylbenzoic acid, ethylbenzoic acid, propylbenzoic acid, butylbenzoic acid, cumic acid, t-butylbenzoic acid, o-toluic acid, m-toluic acid, p-toluic acid, ethoxybenzoic acid, propoxybenzoic acid, naphthoic acid, nicotinic acid, furoic acid, anisic acid, 1-naphthalenecarboxylic acid, 2-naphthalenecarboxylic acid, and methyl esters and acid chlorides thereof. These monocarboxylic acids may be used alone or in combination. Among these, benzoic acid is preferred because the resulting polyester modifier composition has high bleeding resistance at high temperature and humidity and can impart sufficient moisture permeability resistance to optical films.

Among the aromatic polyester resins (A2), the following modifier is an example of polyester modifiers produced from an aliphatic diol having 2 to 4 carbon atoms, an aromatic dicarboxylic acid having 8 to 12 carbon atoms, and an aromatic monocarboxylic acid having 7 to 11 carbon atoms.

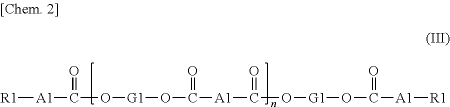

[In the formula (III), R1 independently denotes a hydrogen atom, an alkyl group having 1 to 4 carbon atoms and optionally a side chain, or an alkoxy group having 1 to 4 carbon atoms and optionally a side chain. G1 independently denotes an alkylene group having 2 to 4 carbon atoms and optionally a side chain. A1 independently denotes an aromatic ring structure. n is an integer in the range of 1 to 7.]

The degree of dispersion (Mw/Mn) of the polyester resin (A2) preferably ranges from 1.0 to 3.0, more preferably 1.0 to 1.5. When the polyester resin (A2) has a degree of dispersion in this range, the resulting modifier composition has high compatibility with cellulose ester resins and low volatility.

The polyester resin (A2) preferably has a hydroxyl value in the range of 0 to 20 mgKOH/g, more preferably 0 to 10. The polyester resin (A2) preferably has an acid value in the range of 0 to 1 mgKOH/g, more preferably 0 to 0.5. Thus, the polyester resin (A2) preferably has a hydroxyl value in the range of 0 to 20 mgKOH/g and an acid value in the range of 0 to 1.0 mgKOH/g and more preferably has a hydroxyl value in the range of 0 to 10 and an acid value in the range of 0 to 0.5.

A cellulose ester optical film that contains a modifier composition for a cellulose ester resin according to the present invention and a cellulose ester resin will be described below.

A cellulose ester optical film according to the present invention contains a cellulose ester resin, the modifier composition for a cellulose ester resin, and optionally other various additive agents. The thickness of the film depends on the application and generally preferably ranges from 10 to 200 μm.

The cellulose ester optical film may have optical anisotropy or optical isotropy. When the optical film is used as a polarizing plate protective film, the optical film preferably has optical isotropy, which does not reduce light transmission.

The cellulose ester optical film can be used in various applications. For example, the most effective application is a polarizing plate protective film of a liquid crystal display that requires optical isotropy. The cellulose ester optical film can also be used as a support of a polarizing plate protective film that requires an optical compensation function.

The cellulose ester optical film can be used in liquid crystal cells of various display modes, for example, in-plane switching (IPS), twisted nematic (TN), vertically aligned (VA), and optically compensatory bend (OCB).

The cellulose ester resin contained in the cellulose ester optical film may be a cellulose derived from cotton linters, wood pulp, or kenaf, the hydroxy groups of the cellulose being partly or entirely esterified. Among these, a film formed of a cellulose ester resin produced by esterifying a cellulose derived from cotton linters is preferred because the film can be easily separated from a metal support of a film forming apparatus, thereby further improving film production efficiency.

Examples of the cellulose ester resin include cellulose acetate, cellulose acetate propionate, cellulose acetate butyrate, cellulose acetate phthalate, and cellulose nitrate. When the cellulose ester optical film is used as a polarizing plate protective film, cellulose acetate is preferably used in order to produce a film having good mechanical properties and transparency. These cellulose ester resins may be used alone or in combination.

The degree of polymerization of the cellulose acetate preferably ranges from 250 to 400, and the degree of acetylation of the cellulose acetate preferably ranges from 54.0% to 62.5% by mass, more preferably 58.0% to 62.5% by mass. When the degree of polymerization and the degree of acetylation of the cellulose acetate are in these ranges, the resulting film can have good mechanical properties. In the present invention, cellulose triacetate is more preferably used. The degree of acetylation, as used herein, refers to the mass ratio of acetic acid formed by saponification of cellulose acetate to the total amount of the cellulose acetate.

The cellulose acetate preferably has a Mn in the range of 70,000 to 300,000, more preferably 80,000 to 200,000. When the cellulose acetate has a Mn in this range, the resulting film can have good mechanical properties.

A cellulose ester optical film according to the present invention preferably contains 5 to 30 parts by mass, more preferably 5 to 15 parts by mass, of a modifier composition for a cellulose ester resin according to the present invention per 100 parts by mass of the cellulose ester resin. When the modifier composition for a cellulose ester resin is used in this range, the cellulose ester optical film can have high dimensional stability, high moisture permeability resistance, and good optical properties.

The cellulose ester optical film can be formed by melt-kneading a cellulose ester resin composition containing a cellulose ester resin, a modifier composition for a cellulose ester resin, and optionally other various additive agents in an extruder and forming a film through a T-die.

The cellulose ester optical film can be produced by the forming method described above or a solution casting method (solvent casting method), which includes casting a resin solution on a metal support, the resin solution produced by dissolving the cellulose ester resin and the modifier composition for a cellulose ester resin in an organic solvent, and then evaporating the organic solvent to dry the resin solution.

In the solution casting method, the orientation of the cellulose ester resin can be restricted during the formation of the film, and the resulting film substantially has optical isotropy. The film having optical isotropy can be used as an optical material for liquid crystal displays and is particularly useful as a polarizing plate protective film. The film produced by the method has fewer irregularities on its surface and has surface smoothness.

In general, the solution casting method includes a first step of dissolving the cellulose ester resin and the modifier composition for a cellulose ester resin in an organic solvent and casting the resin solution on a metal support, a second step of evaporating the organic solvent from the casted resin solution to dry the resin solution and form a film, and a subsequent third step of separating the film from the metal support and heat-drying the film.

The metal support used in the first step may be an endless belt or drum-shaped metallic support. For example, the metal support is a stainless steel support having a mirror-finished surface.

Before the resin solution is casted on the metal support, the resin solution is preferably passed through a filter in order to avoid contamination of the film with foreign matter.

The drying method in the second step is not particularly limited. For example, air having a temperature in the range of 30° C. to 50° C. is blown over the top and/or bottom surface of the metal support to evaporate 50% to 80% by mass of the organic solvent in the casted resin solution, thereby forming a film on the metal support.

Then, in the third step, the film formed in the second step is separated from the metal support and is heat-dried at a higher temperature than in the second step. The heat-drying method may preferably be a method for increasing the temperature stepwise at a temperature in the range of 100° C. to 160° C. in terms of dimensional stability. Heat-drying under the temperature condition can almost entirely remove the organic solvent remaining in the film after the second step.

In the first to third steps, the organic solvent may be collected and reused.

The organic solvent for mixing and dissolving the cellulose ester resin and the modifier composition for a cellulose ester resin may be any organic solvent that can dissolve them. For example, when the cellulose ester is cellulose acetate, an organic halide, such as methylene chloride, or a dioxolane is preferably used as a good solvent.

In combination with the good solvent, a poor solvent, such as methanol, ethanol, 2-propanol, n-butanol, cyclohexane, or cyclohexanone, is preferably used to improve film production efficiency.

The mixing ratio of the good solvent to the poor solvent preferably ranges from 75/25 to 95/5 on a mass basis.

The concentration of the cellulose ester resin in the resin solution preferably ranges from 10% to 50% by mass, more preferably 15% to 35% by mass.

Various additive agents may be used in the cellulose ester optical film without compromising the object of the present invention.

Examples of the additive agents include modifiers other than a modifier composition for a cellulose ester resin according to the present invention, thermoplastic resins, ultraviolet absorbers, matting agents, antidegradants (for example, antioxidants, peroxide decomposers, radical inhibitors, metal deactivators, and acid scavengers), and dyes. These additive agents may be used separately or in combination when the cellulose ester resin and the modifier for a cellulose ester resin are dissolved and mixed in the organic solvent.

Examples of the modifiers other than the modifier composition for a cellulose ester resin include phosphates, such as triphenyl phosphate (TPP), tricresyl phosphate, and cresyl diphenyl phosphate, phthalate esters, such as dimethyl phthalate, diethyl phthalate, dibutyl phthalate, and di-2-ethylhexyl phthalate, ethyl phthalyl ethyl glycolate, butyl phthalyl butyl glycolate, trimethylolpropane tribenzoate, pentaerythritol tetraacetate, and acetyl tributyl citrate.

Examples of the thermoplastic resins include, but are not limited to, polyester resins other than polyesters contained in a modifier composition for a cellulose ester resin according to the present invention, polyester-ether resins, polyurethane resins, epoxy resins, and toluenesulfonamide resins.

Examples of the ultraviolet absorbers include, but are not limited to, oxybenzophenone compounds, benzotriazole compounds, salicylate compounds, benzophenone compounds, cyanoacrylate compounds, and nickel complex salt compounds. The amount of ultraviolet absorber preferably ranges from 0.01 to 2 parts by mass per 100 parts by mass of the cellulose ester resin.

Examples of the matting agents include silicon oxide, titanium oxide, aluminum oxide, calcium carbonate, calcium silicate, aluminum silicate, magnesium silicate, calcium phosphate, kaolin, and talc. The amount of matting agent preferably ranges from 0.1 to 0.3 parts by mass per 100 parts by mass of the cellulose ester resin.

The dyes may be of any type and may be used in any amount, provided that the object of the present invention is not compromised.

Because of its high moisture permeability resistance, transparency, and optical anisotropy in the thickness direction, a cellulose ester optical film according to the present invention can be used as an optical film of liquid crystal displays, for example. Examples of the optical film of liquid crystal displays include polarizing plate protective films, retardation films, reflective films, viewing angle improving films, antiglare films, antireflection films, antistatic films, and color filters. Among these, a cellulose ester optical film according to the present invention can be preferably used as a polarizing plate protective film.

The cellulose ester optical film preferably has a thickness in the range of 20 to 120 μm, more preferably 25 to 100 μm, particularly preferably 25 to 80 μm. When the optical film is used as a polarizing plate protective film, the optical film preferably has a thickness in the range of 25 to 80 μm in order to decrease the thickness of the liquid crystal display while maintaining sufficient film strength, Rth stability, and moisture permeability resistance.

The polarizing plate protective film does not cause bleeding at high temperature and humidity and can be adjusted for a desired Rth. Thus, the polarizing plate protective film can be widely used in various liquid crystal display methods depending on the intended use.

### EXAMPLES

The present invention will be more specifically described in the following examples. Unless otherwise specified, parts and % in the examples are based on mass.

### Example 1

### Preparation of Polyester Modifier Composition for Cellulose Ester Resin According to Present Invention

A four-neck flask having an internal volume of 2 liters equipped with a thermometer, a stirrer, and a reflux condenser was charged with 404 g of 1,2-propylene glycol as a diol, 79 g of adipic acid and 240 g of phthalic anhydride as dicarboxylic acids, 586 g of benzoic acid as a monocarboxylic acid, and 0.079 g of tetraisopropyl titanate as an esterification catalyst. The mixture was heated stepwise to 230° C. in a nitrogen stream while stirring and was allowed to react at 230° C. After a dehydration condensation reaction for 19 hours in total, a reactant (oxidation: 0.22, hydroxy group: 16) was obtained. The reactant had a number-average molecular weight (Mn) of 420. The amount of polyester resin having a molecular weight of less than 350 was 33.0% by mass [the reactant is hereinafter referred to as a comparative modifier composition for a cellulose ester resin (1′)]. The comparative modifier composition for a cellulose ester resin (1′) was distilled in a thin-film distillation apparatus (a thin-film molecule distillation apparatus AS-MDA-65FJ-S manufactured by Asahi Glassplant Inc.) at a distillation tube temperature of 180° C., at a feed tube temperature of 100° C., at a condenser temperature of 40° C., and at a degree of vacuum of 0.012 Pa, thus yielding a polyester modifier composition for a cellulose ester resin (1) according to the present invention. The modifier composition (1) had a number-average molecular weight (Mn) of 590. The amount of polyester resin having a molecular weight of less than 350 was 2.0% by mass.

### Example 2

### Same as Above

A four-neck flask having an internal volume of 2 liters equipped with a thermometer, a stirrer, and a reflux condenser was charged with 356 g of 1,2-propylene glycol as a diol, 393 g of dimethylterephthalic acid as a dicarboxylic acid, 581 g of paratoluic acid as a monocarboxylic acid, and 0.079 g of tetraisopropyl titanate as an esterification catalyst. The mixture was heated stepwise to 230° C. in a nitrogen stream while stirring and was allowed to react at 230° C. After a dehydration condensation reaction for 17 hours in total, a reactant (oxidation: 0.21, hydroxy group: 9) was obtained. The reactant had a number-average molecular weight (Mn) of 480. The amount of polyester resin having a molecular weight of less than 350 was 34.0% by mass [the reactant is hereinafter referred to as a comparative modifier composition for a cellulose ester resin (2′)]. The comparative modifier composition for a cellulose ester resin (2′) was distilled in the thin-film distillation apparatus at a distillation tube temperature of 180° C., at a feed tube temperature of 100° C., at a condenser temperature of 40° C., and at a degree of vacuum of 0.012 Pa, thus yielding a polyester modifier composition for a cellulose ester resin (2) according to the present invention. The modifier composition (2) had a number-average molecular weight (Mn) of 620. The amount of polyester resin having a molecular weight of less than 350 was 3.8% by mass.

### Example 3

### Same as Above

A four-neck flask having an internal volume of 2 liters equipped with a thermometer, a stirrer, and a reflux condenser was charged with 410 g of 1,2-propylene glycol as a diol, 463 g of dimethylterephthalic acid as a dicarboxylic acid, 648 g of benzoic acid as a monocarboxylic acid, and 0.091 g of tetraisopropyl titanate as an esterification catalyst. The mixture was heated stepwise to 230° C. in a nitrogen stream while stirring and was allowed to react at 230° C. After a dehydration condensation reaction for 15 hours in total, a reactant (oxidation: 0.1, hydroxy group: 5) was obtained. The reactant had a number-average molecular weight (Mn) of 450. The amount of polyester resin having a molecular weight of less than 350 was 26.0% by mass [the reactant is hereinafter referred to as a comparative modifier composition for a cellulose ester resin (3′)]. The comparative modifier composition for a cellulose ester resin (3′) was distilled in the thin-film distillation apparatus at a distillation tube temperature of 180° C., at a feed tube temperature of 100° C., at a condenser temperature of 40° C., and at a degree of vacuum of 0.012 Pa, thus yielding a polyester modifier composition for a cellulose ester resin (3) according to the present invention. The modifier composition (3) had a number-average molecular weight (Mn) of 630. The amount of polyester resin having a molecular weight of less than 350 was 2.0% by mass.

### Example 4

### Same as Above

A four-neck flask having an internal volume of 2 liters equipped with a thermometer, a stirrer, and a reflux condenser was charged with 355 g of ethylene glycol as a diol, 645 g of adipic acid as a dicarboxylic acid, and 0.030 g of tetraisopropyl titanate as an esterification catalyst. The mixture was heated stepwise to 220° C. in a nitrogen stream while stirring and was allowed to react at 220° C. After a dehydration condensation reaction for 15 hours in total, a reactant (acid value: 0.3, hydroxy value: 140) was obtained. The reactant had a number-average molecular weight (Mn) of 1000. The amount of polyester resin having a (Mn) of less than 350 was 7.0% by mass [the reactant is hereinafter referred to as a comparative modifier composition for a cellulose ester resin (4′)]. The comparative modifier composition for a cellulose ester resin (4′) was distilled in the thin-film distillation apparatus at a distillation tube temperature of 200° C., at a feed tube temperature of 90° C., at a condenser temperature of 40° C., and at a degree of vacuum of 0.012 Pa, thus yielding a polyester modifier composition for a cellulose ester resin (4) according to the present invention. The modifier composition (4) had a number-average molecular weight (Mn) of 1310. The amount of polyester resin having a (Mn) of less than 350 was 2.4% by mass.

### Example 5

### Same as Above

A three-neck flask having an internal volume of 1 liter equipped with a thermometer, a stirrer, and a reflux condenser was charged with 217 g of ethylene glycol as a diol, 208 g of 1,2-dicarboxycyclohexane and 372 g of succinic acid as dicarboxylic acids, 163 g of n-butanol as a monoalcohol, and 0.03 g of tetraisopropyl titanate as an esterification catalyst. The mixture was heated stepwise to 220° C. in a nitrogen stream while stirring and was allowed to react at 220° C. After a dehydration condensation reaction for 30 hours in total, a reactant (oxidation: 0.43, hydroxy group: 5.4) was obtained. The reactant had a number-average molecular weight (Mn) of 820. The amount of polyester resin having a molecular weight of less than 350 was 16% by mass [the reactant is hereinafter referred to as a comparative modifier composition for a cellulose ester resin (5′)]. The comparative modifier composition for a cellulose ester resin (5′) was distilled in the thin-film distillation apparatus at a distillation tube temperature of 200° C., at a feed tube temperature of 90° C., at a condenser temperature of 40° C., and at a degree of vacuum of 0.012 Pa, thus yielding a polyester modifier composition for a cellulose ester resin (5) according to the present invention. The modifier composition (5) had a number-average molecular weight (Mn) of 1010. The amount of polyester resin having a molecular weight of less than 350 was 1.8% by mass.

### Example 6

### Preparation of Cellulose Ester Optical Film According to Present Invention

A doping liquid was prepared by dissolving 100 parts of a cellulose triacetate resin (“LT-35” manufactured by Daicel Chemical Industries, Ltd.) and 10 parts of the modifier composition for a cellulose ester resin (1) in a mixed solvent of 810 parts of methylene chloride and 90 parts of methanol. The doping liquid was casted on a glass plate at a thickness of 0.8 mm and was dried at room temperature for 16 hours, at 50° C. for 30 minutes, and at 120° C. for 30 minutes, thereby forming a cellulose ester optical film (1) according to the present invention. The film (1) had a thickness of 60 μm.

### Example 7

### Same as Above

A cellulose ester optical film (2) was formed in the same manner as in Example 6 except that the modifier composition for a cellulose ester resin (1) was substituted by the modifier composition for a cellulose ester resin (2).

### Example 8

### Same as Above

A cellulose ester optical film (3) was formed in the same manner as in Example 6 except that the modifier composition for a cellulose ester resin (1) was substituted by the modifier composition for a cellulose ester resin (3).

### Example 9

### Same as Above

A cellulose ester optical film (4) was formed in the same manner as in Example 6 except that the modifier composition for a cellulose ester resin (1) was substituted by the modifier composition for a cellulose ester resin (4).

### Example 10

### Same as Above

A cellulose ester optical film (5) was formed in the same manner as in Example 6 except that the modifier composition for a cellulose ester resin (1) was substituted by the modifier composition for a cellulose ester resin (5).

### Comparative Example 1

### Preparation of Comparative Cellulose Ester Optical Film

A cellulose ester optical film (1′) was formed in the same manner as in Example 6 except that the modifier composition for a cellulose ester resin (1) was substituted by the comparative modifier composition for a cellulose ester resin (1′).

### Comparative Example 2

### Same as Above

A cellulose ester optical film (2′) was formed in the same manner as in Example 6 except that the modifier composition for a cellulose ester resin (1) was substituted by the comparative modifier composition for a cellulose ester resin (2′).

### Comparative Example 3

### Same as Above

A cellulose ester optical film (3′) was formed in the same manner as in Example 6 except that the modifier composition for a cellulose ester resin (1) was substituted by the comparative modifier composition for a cellulose ester resin (3′).

### Comparative Example 4

### Same as Above

A cellulose ester optical film (4′) was formed in the same manner as in Example 6 except that the modifier composition for a cellulose ester resin (1) was substituted by the comparative modifier composition for a cellulose ester resin (4′).

### Comparative Example 5

### Same as Above

A cellulose ester optical film (5′) was formed in the same manner as in Example 6 except that the modifier composition for a cellulose ester resin (1) was substituted by the comparative modifier composition for a cellulose ester resin (5′).

### Test Example 1

### Evaluation of Dimensional Stability of Cellulose Ester Film

Dimensional stability was evaluated by the following method in the cellulose ester optical film (1) formed from the modifier composition for a cellulose ester resin (1) according to the present invention and the comparative cellulose ester optical film (1′) formed from the comparative cellulose ester resin composition (1′), which is the same raw material as the modifier composition for a cellulose ester resin (1).

### <Method for Evaluating Dimensional Stability>

The rate of change in the dimensions of an optical film exposed to a heating environment was measured. More specifically, first, the dimensions of a cellulose ester optical film in the MD direction (the film forming direction) and TD direction (a direction perpendicular to the film forming direction) before exposure to the heating environment were measured with a CNC image measuring apparatus NEXIV VMR-6555 (manufactured by Nikon Instech Co., Ltd.). After measurement, the cellulose ester optical film was left at a temperature of 140° C. and at a humidity of 0% for 45 minutes. After that, the dimensions of the optical film in the MD and TD directions were measured with the CNC image measuring apparatus. The rate of change in dimensions due to exposure to the heating environment was determined in the MD and TD directions. The rates of change were averaged for the evaluation of the dimensional change rate. A positive dimensional change rate indicates that the dimensions of the film are greater after exposure to the heating environment than before exposure to the heating environment. A negative dimensional change rate indicates that the dimensions of the film are smaller after exposure to the heating environment than before exposure to the heating environment. An optical film having a dimensional change rate closer to zero has higher dimensional stability.

After the cellulose ester optical film (1) was left in the heating environment, the dimensions of the cellulose ester optical film (1) in the TD and MD directions measured by the evaluation method were decreased by 0.29% on average. Thus, the dimensional change rate based on the evaluation method is −0.29%. The dimensions of the comparative cellulose ester optical film (1′) in the TD and MD directions were decreased by 0.437% on average. Thus, the dimensional change rate based on the evaluation method is −0.437%. On the basis of the dimensional change rate of the comparative cellulose ester optical film (1′), the dimensional change rate of the cellulose ester optical film (1) was improved by [(0.437−0.29)/0.437]×100=30.6%.

### Test Example 2

### Same as Above

Dimensional stability was evaluated in the same manner as in Test Example 1 except for use of the cellulose ester optical film (2) formed from the modifier composition for a cellulose ester resin (2) according to the present invention and the comparative cellulose ester optical film (2′) formed from the comparative cellulose ester resin composition (2′), which is the same raw material as the modifier composition for a cellulose ester resin (2).

After the cellulose ester optical film (2) was left in the heating environment, the dimensions of the cellulose ester optical film (2) in the TD and MD directions measured by the evaluation method were decreased by 0.344% on average. Thus, the dimensional change rate based on the evaluation method is −0.344%. The dimensions of the comparative cellulose ester optical film (2′) in the TD and MD directions were decreased by 0.402% on average. Thus, the dimensional change rate based on the evaluation method is −0.402%. On the basis of the dimensional change rate of the comparative cellulose ester optical film (2′), the dimensional change rate of the cellulose ester optical film (2) was improved by [(0.402−0.344)/0.402]×100=14.4%.

### Test Example 3

### Same as Above

Dimensional stability was evaluated in the same manner as in Test Example 1 except for use of the cellulose ester optical film (3) formed from the modifier composition for a cellulose ester resin (3) according to the present invention and the comparative cellulose ester optical film (3′) formed from the comparative cellulose ester resin composition (3′), which is the same raw material as the modifier composition for a cellulose ester resin (3).

After the cellulose ester optical film (3) was left in the heating environment, the dimensions of the cellulose ester optical film (3) in the TD and MD directions measured by the evaluation method were decreased by 0.410% on average. Thus, the dimensional change rate based on the evaluation method is −0.410%. The dimensions of the comparative cellulose ester optical film (3′) in the TD and MD directions were decreased by 0.487% on average. Thus, the dimensional change rate based on the evaluation method is −0.487%. On the basis of the dimensional change rate of the comparative cellulose ester optical film (3′), the dimensional change rate of the cellulose ester optical film (3) was improved by [(0.487−0.410)/0.487]×100=15.8%.

### Test Example 4

### Same as Above

Dimensional stability was evaluated in the same manner as in Test Example 1 except for use of the cellulose ester optical film (4) formed from the modifier composition for a cellulose ester resin (4) according to the present invention and the comparative cellulose ester optical film (4′) formed from the comparative cellulose ester resin composition (4′), which is the same raw material as the modifier composition for a cellulose ester resin (4).

After the cellulose ester optical film (4) was left in the heating environment, the dimensions of the cellulose ester optical film (4) in the TD and MD directions measured by the evaluation method were decreased by 0.380% on average. Thus, the dimensional change rate based on the evaluation method is −0.380%. The dimensions of the comparative cellulose ester optical film (4′) in the TD and MD directions were decreased by 0.420% on average. Thus, the dimensional change rate based on the evaluation method is −0.420%. On the basis of the dimensional change rate of the comparative cellulose ester optical film (4′), the dimensional change rate of the cellulose ester optical film (4) was improved by [(0.420−0.380)/0.420]×100=9.5%.

### Test Example 5

### Same as Above

Dimensional stability was evaluated in the same manner as in Test Example 1 except for use of the cellulose ester optical film (5) formed from the modifier composition for a cellulose ester resin (5) according to the present invention and the comparative cellulose ester optical film (5′) formed from the comparative cellulose ester resin composition (5′), which is the same raw material as the modifier composition for a cellulose ester resin (5).

After the cellulose ester optical film (5) was left in the heating environment, the dimensions of the cellulose ester optical film (5) in the TD and MD directions measured by the evaluation method were decreased by 0.382% on average. Thus, the dimensional change rate based on the evaluation method is −0.382%. The dimensions of the comparative cellulose ester optical film (5′) in the TD and MD directions were decreased by 0.485% on average. Thus, the dimensional change rate based on the evaluation method is −0.485%. On the basis of the dimensional change rate of the comparative cellulose ester optical film (5′), the dimensional change rate of the cellulose ester optical film (5) was improved by [(0.485−0.382)/0.485]×100=21.2%.

1-11. (canceled)

12. A polyester modifier composition for a cellulose ester resin, comprising: a polyester resin produced by a reaction between a diol and 1,2-dicarboxycyclohexane, wherein the modifier composition has a number-average molecular weight (Mn) in the range of 350 to 2,000 as measured by gel permeation chromatography (GPC), and a polyester resin having a molecular weight of less than 350 constitutes 5% or less by mass of the modifier composition.

13. The polyester modifier composition for a cellulose ester resin according to claim 12, wherein the modifier composition has a number-average molecular weight (Mn) in the range of 500 to 1,800 as measured by gel permeation chromatography (GPC), and the polyester resin having a molecular weight of less than 350 constitutes 3% or less by mass of the modifier composition.

14. The polyester modifier composition for a cellulose ester resin according to claim 12, wherein after a polyester resin composition is produced by a reaction between the diol and 1,2-dicarboxycyclohexane, the polyester resin having a molecular weight of less than 350 is removed from the polyester resin composition by thin film distillation to produce the polyester modifier composition.

15. The polyester modifier composition for a cellulose ester resin according to claim 12, wherein the polyester resin is a polyester resin produced by a reaction between the aliphatic diol having 2 to 4 carbon atoms, 1,2-dicarboxycyclohexane, and a monoalcohol having 4 to 9 carbon atoms.

16. The polyester modifier composition for a cellulose ester resin according to claim 12, wherein the polyester resin is a polyester resin produced by a reaction between the aliphatic diol having 2 to 4 carbon atoms, 1,2-dicarboxycyclohexane, and a monocarboxylic acid having 4 to 9 carbon atoms.

17. A cellulose ester optical film, comprising: the polyester modifier composition for a cellulose ester resin according to claim 12; and a cellulose ester resin.

18. The cellulose ester optical film according to claim 17, containing 5 to 30 parts by mass of the polyester modifier composition for a cellulose ester resin per 100 parts by mass of the cellulose ester resin.

19. A polarizing plate protective film, formed by casting a resin solution on a metal support, the resin solution produced by dissolving the polyester modifier composition for a cellulose ester resin according to claim 12 and a cellulose ester resin in an organic solvent, and then evaporating the organic solvent to dry the resin solution.

20. A polyester modifier composition for a cellulose ester resin, comprising: a polyester resin produced by a reaction between 1,2-propylene glycol, adipic acid, phthalic anhydride, and benzoic acid, wherein the modifier composition has a number-average molecular weight (Mn) in the range of 350 to 2,000 as measured by gel permeation chromatography (GPC), and a polyester resin having a molecular weight of less than 350 constitutes 5% or less by mass of the modifier composition.

21. The polyester modifier composition for a cellulose ester resin according to claim 20, wherein the modifier composition has a number-average molecular weight (Mn) in the range of 500 to 1,800 as measured by gel permeation chromatography (GPC), and the polyester resin having a molecular weight of less than 350 constitutes 3% or less by mass of the modifier composition.

22. The polyester modifier composition for a cellulose ester resin according to claim 20, wherein after a polyester resin composition is produced by a reaction between 1,2-propylene glycol, adipic acid, phthalic anhydride, and benzoic acid, the polyester resin having a molecular weight of less than 350 is removed from the polyester resin composition by thin film distillation to produce the polyester modifier composition.

23. A cellulose ester optical film, comprising: the polyester modifier composition for a cellulose ester resin according to claim 20; and a cellulose ester resin.

24. The cellulose ester optical film according to claim 23, containing 5 to 30 parts by mass of the polyester modifier composition for a cellulose ester resin per 100 parts by mass of the cellulose ester resin.

25. A polarizing plate protective film, formed by casting a resin solution on a metal support, the resin solution produced by dissolving the polyester modifier composition for a cellulose ester resin according to claim 20 and a cellulose ester resin in an organic solvent, and then evaporating the organic solvent to dry the resin solution.
